(19) United States
(12) Patent Application Publication
(10) Pub. No.: US 2025/0066444 A1
(43) Pub. Date: Feb. 27, 2025

(54) GLUCAGON PROMOTER FOR DIABETES GENE THERAPY

(71) Applicant: University of Pittsburgh - Of the Commonwealth System of Higher Education, Pittsburgh, PA (US)

(72) Inventors: George Gittes, Pittsburgh, PA (US); Ting Zhang, Glenshaw, PA (US)

(73) Assignee: University of Pittsburgh - Of the Commonwealth System of Higher Education, Pittsburgh, PA (US)

(21) Appl. No.: 18/726,764

(22) PCT Filed: Jan. 3, 2023
(86) PCT No.: PCT/US2023/060036

§ 371 Date: Jul. 3, 2024

Related U.S. Application Data
(60) Provisional application No. 63/296,432, filed on Jan. 4, 2022.

(51) Int. Cl. C07K 14/605; A61K 48/00; C07K 14/47; C12N 15/86

(52) U.S. Cl. CPC C07K 14/605; A61K 48/0075; C07K 14/4702; C12N 15/86; C12N 2750/14143

(57) ABSTRACT

Glucagon promoters are disclosed herein. In addition, disclosed are recombinant nucleic acid molecules that include a glucagon promoter operably linked to a nucleic acid molecule encoding at least one heterologous protein. Vectors including these recombinant nucleic acid molecules, and host cells transformed with these vectors, are also disclosed. Methods of use for these promoters, recombinant nucleic acid molecules, vectors and host cells are disclosed.

### CROSS REFERENCE TO RELATED APPLICATIONS

This claims the benefit of U.S. Provisional Application No. 63/296,432, filed Jan. 4, 2022, which is incorporated herein by reference.

### ACKNOWLEDGEMENT OF GOVERNMENT SUPPORT

This invention was made with government support under grant nos. DK120377 and DK112836 awarded by the National Institutes of Health. The government has certain rights in the invention.

### FIELD OF THE DISCLOSURE

This relates to the field of alpha cells, specifically to glucagon promoters that can be used to express a heterologous protein in alpha cells.

### REFERENCE TO AN ELECTRONIC SEQUENCE LISTING

The electronic sequence listing (8123-107679-02 sequence listing 18.xml; Size: 20000 bytes; and Date of Creation: Dec. 13, 2022) is herein incorporated by reference in its entirety.

### BACKGROUND

A mammalian pancreas is composed of two subclasses of tissue: the exocrine cells of the acinar tissue and the endocrine cells of the islets of Langerhans. The exocrine cells produce digestive enzymes that are secreted through the pancreatic duct to the intestine. The islet cells produce polypeptide hormones that are involved in carbohydrate metabolism. The islands of endocrine tissue that exist within the adult mammalian pancreas are termed the islets of Langerhans. Adult mammalian islets are composed of five major cell types, the alpha (u), beta ((3), delta (6), pancreatic polypeptide (PP), and ghrelin (F) cells. These cells are distinguished by their production of glucagon, insulin, somatostatin, pancreatic polypeptide, and ghrelin, respectively.

PCT Publication No. WO 2015/164218, incorporated herein by reference, discloses that adeno-associated virus encoding PDX1 and MAFA can be infused through the pancreatic duct, such as by using endoscopic retrograde cholangiopancreatography (ERCP), to transdifferentiate alpha-cells into functional beta-cells. However, there remains a need for promoters that can be used to achieve high levels of these proteins, and/or other proteins, in alpha cells.

### SUMMARY OF THE DISCLOSURE

Glucagon promoters are disclosed herein, wherein the glucagon promoter consists essentially of, or consists of: a nucleotide sequence at least 95% identical to SEQ ID NO: 1, that functions as a promoter; the nucleotide sequence of SEQ ID NO: 1; a nucleotide sequence at least 95% identical to SEQ ID NO: 2, that functions as a promoter; the nucleotide sequence of SEQ ID NO: 2; a nucleotide sequence at least 95% identical to SEQ ID NO: 3, that functions as a promoter; or the nucleotide sequence of SEQ ID NO: 3.

In further embodiments, recombinant nucleic acid molecules are disclosed that include a glucagon promoter operably linked to a heterologous nucleic acid molecule encoding a protein. Also disclosed are vectors include these recombinant nucleic acid molecules, and host cells transformed with these vectors.

In more embodiments, methods of using these vectors and host cells are disclosed. Methods for treating diabetes in a subject are also provided.

The foregoing and other features and advantages of the invention will become more apparent from the following detailed description of several embodiments which proceeds with reference to the accompanying figures.

### BRIEF DESCRIPTION OF THE FIGURES

FIG. 1. Human glucagon promoter region map. Adapted from USCS Genome Browser. In the human glucagon promoter region, the TATA box and CAAT box are located at −20 to −26 and −62 to −65, respectively. FoxA1 binds just upstream of the TATA box. FoxA proteins play a role in direct transcriptional activation and may also play a role in opening the chromatin at the start site of transcription of the glucagon gene. Pax6 binds the G1 region; NFAT1B binds the G2 region; Pax6 and Prep/Pbx bind the G3 region; and CREB binds upstream of the G3 region. The G1 region, characterized by low intrinsic transcriptional activity, is critical for specific expression of the glucagon gene in alpha cells. The other two conserved regions G2 and G3, independently display enhancer-like functions on both homologous and heterologous promoters in glucagon (alpha) cells, but only on heterologous promoters in insulin (beta) and somatostatin (delta) expressing cells, and not in non-islet cells. These phenotypically distinct islet cell types all contain regulatory DNA-binding proteins that interact with the three control elements of the glucagon gene. The cell-specific DNA-binding proteins, when interacting with the glucagon promoter, only result in transcriptional activation in alpha cells. In the non-alpha cells there is glucagon gene repression.

FIG. 2. Mouse glucagon promoter region map. Adapted from USCS Genome Browser. In the mouse glucagon promoter region, the TATA box and CAAT box are located at −20 to −24 and −64 to −67. Alx, Pax6 and PDX1 bind the G1 region; Pax6 and Alx3 bind the G3 region, and ATF3 binds the CRE/ATF motif. These transcription factors interact with the glucagon promoter resulting in transcriptional activation in mouse alpha cells

FIG. 3. Schema of pAAV-hGCGp592 bp-hPM-GFP, pAAV-hGCGp660 bp-hPM-GFP, and pAAV-mGcgp515 bp-hPM-GFP vectors.

FIG. 4. Glucagon promoter driven human PDX1, human MAFA, and GFP transduction in human alpha cells by plasmid transfection. Human alpha cells were transfected by a plasmid containing a CMV promoter driving human PDX1, MAFA, and GFP (hPM-GFP). The human glucagon promoter 592 bp driving hPM-GFP showed a stronger expression as measured by GFP compared with the CMV promoter control. The human glucagon promoter 660 bp driving hPM-GFP showed a similar expression of GFP as the CMV control. The mouse glucagon promoter 515 bp driving hPM-GFP showed less expression of GFP than the CMV control.

FIG. 5. Secreted insulin levels in human alpha cell culture media after transduction with different plasmids. Secreted insulin levels in the culture media were measured by insulin ELISA. Transduction by the disclosed glucagon promoter driving human PDX1, human MAFA, and GFP showed a 7-fold greater amount of insulin secretion into the culture media compared with the CMV promoter driving hPM and GFP. Three repetitions were performed with similar results. This 7-fold increase is highly significant.

### SEQUENCES

The nucleic and amino acid sequences listed are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NOs: 1-3 are nucleic acid sequences of glucagon promoters.

SEQ ID NO: 4 is an exemplary amino acid sequence of human MAFA.

SEQ ID NO: 5 is an exemplary amino acid sequence of mouse MAFA.

SEQ ID NO: 6 is an exemplary amino acid sequence of human PDX1.

SEQ ID NO: 7 is an exemplary amino acid sequence of mouse PDX1.

SEQ ID NO: 8 is the nucleic acid sequence of the 2A connector.

SEQ ID NOs: 9-10 are the nucleic acid sequences of connectors.

SEQ ID NO: 11 is an exemplary amino acid sequence of human Ngn3.

SEQ ID NOs: 12-18 are nucleic acid sequences of a domain of a disclosed glucagon promoter.

### DETAILED DESCRIPTION OF SEVERAL EMBODIMENTS

Disclosed herein are several glucagon promoters that can be operably linked to a heterologous nucleic acid molecule encoding a protein. Recombinant nucleic acid molecules including such a glucagon promoter operably linked to the heterologous nucleic acid molecule encoding a protein provide a high level of expression of the protein in alpha cells. Vectors, such as adeno-associated virus vectors including a disclosed glucagon promoter operably linked to a heterologous nucleic acid molecule encoding Pdx1 and MafA can be infused through the pancreatic duct, such as by using endoscopic retrograde cholangiopancreatography (ERCP), to transdifferentiate alpha-cells into functional beta-cells.

PCT Publication No. WO 2015/164218, incorporated herein by reference, discloses that adeno-associated virus encoding PDX1 and MAFA can be infused through the pancreatic duct, such as by using endoscopic retrograde cholangiopancreatography (ERCP), to reprogram alpha-cells into functional beta-cells. The new beta cells are immunologically unrecognized for an extended period, resulting in persistent euglycemia without further interventions. In addition, the immune system in treated subjects was reverted to a naive state in which the immune cells were not actively being exposed to beta-cell autoantigens. The presently disclosed promoters can be used to achieve high levels of expression of MAFA and PDX1 and can be used to increase the efficiency of these methods.

### Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references. As used herein, the singular forms “a,” “an,” and “the,” refer to both the singular as well as plural, unless the context indicates otherwise. For example, the term “a beta cell” includes single or plural beta cells and can be considered equivalent to the phrase “at least one beta cell.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. To facilitate review of the various embodiments, the following explanations of terms are provided:

Alpha (α) cells: Mature glucagon producing endocrine cells. In vivo, these cells are found in the pancreatic islets of Langerhans.

Beta (β) cells: Mature insulin producing endocrine cells. In vivo, these cells are found in the pancreatic islets of Langerhans,

Delta (δ) cells: Mature somatostatin producing endocrine cells. In vivo, these cells are found in the pancreatic islets of Langerhans.

PP cells: Mature pancreatic polypeptide (PP) producing endocrine cells. In vivo, these cells are found in the pancreatic islets of Langerhans.

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 14 recognized serotypes of AAV (AAV1-13 and AAV-DJ).

Administration: To provide or give a subject an agent by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes. In some embodiments, administration is to a pancreatic duct.

Agent: Any polypeptide, compound, small molecule, organic compound, salt, polynucleotide, vector, or other molecule of interest. Agent can include a therapeutic agent, a diagnostic agent or a pharmaceutical agent. A therapeutic agent is a substance that demonstrates some therapeutic effect by restoring or maintaining health, such as by alleviating the symptoms associated with a disease or physiological disorder, or delaying (including preventing) progression or onset of a disease, such as T1D. A therapeutic agent can include a disclosed glucagon promoter operably linked to a nucleic acid molecule encoding a polypeptide.

Animal: Living multi-cellular vertebrate organisms, a category that includes, for example, mammals and birds. The term mammal includes both human and non-human mammals. Similarly, the term “subject” includes both human and veterinary subjects.

Anti-diabetic lifestyle modifications: Changes to lifestyle, habits, and practices intended to alleviate the symptoms of diabetes or pre-diabetes. Obesity and sedentary lifestyle may both independently increase the risk of a subject developing type II diabetes, so anti-diabetic lifestyle modifications include those changes that will lead to a reduction in a subject's body mass index (BMT), increase physical activity, or both. Specific, non-limiting examples include the lifestyle interventions described in Diabetes Care, 22(4):623-34 at pages 626-27, herein incorporated by reference.

Conservative Substitutions: Modifications of a polypeptide that involve the substitution of one or more amino acids for amino acids having similar biochemical properties that do not result in change or loss of a biological or biochemical function of the polypeptide are designated “conservative” substitutions. These conservative substitutions are likely to have minimal impact on the activity of the resultant protein. Table 1 shows amino acids that can be substituted for an original amino acid in a protein, and which are regarded as conservative substitutions.

TABLE

| Original Residue | Conservative Substitutions |
|---|---|
| Ala | ser |
| Arg | lys |
| Asn | gln; his |
| Asp | glu |
| Cys | ser |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

One or more conservative changes, or up to ten conservative changes (such as two substituted amino acids, three substituted amino acids, four substituted amino acids, or five substituted amino acids, etc.) can be made in the polypeptide without changing a biochemical function of the protein, such as PDX1 or MAFA.

Consists essentially of: With regarding to nucleic acid molecules, a term that indicates that additional nucleotides are not included in the part of the molecule, but that other agents (such as labels, altered forms of a nucleotide, or chemical compounds) can be included. “Consists of” indicates that the exact nucleotide sequence is present in that part of a molecule.

Diabetes mellitus: A group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 1 diabetes mellitus is characterized by loss of the insulin-producing βcells, leading to insulin deficiency. This type can be further classified as immune-mediated or idiopathic. Type 2 diabetes results from insulin resistance, a condition in which cells fail to use insulin properly, sometimes combined with an absolute insulin deficiency. This form is also called “non insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and is diagnosed by demonstrating any one of:
a. Fasting plasma glucose level≥7.0 mmol/l (126 mg/dl);
b. Plasma glucose≥11.1 mmol/l (200 mg/dL) two hours after a 75 g oral glucose load as in a glucose tolerance test;
c. Symptoms of hyperglycemia and casual plasma glucose≥11.1 mmol/l (200 mg/dl);
d. Glycated hemoglobin (Hb A1C)≥6.5%

Differentiation: The process whereby a first cell acquires specialized structural and/or functional features characteristic of a certain type of mature cells. Similarly, “differentiate” refers to this process. Typically, during differentiation, cellular structure alters and tissue-specific proteins appear. The term “differentiated pancreatic endocrine cell” refers to cells expressing a protein characteristic of the specific pancreatic endocrine cell type. A differentiated pancreatic endocrine cell includes an α cell, a β cell, a δ cell, and a PP cell, which express glucagon, insulin, somatostatin, and pancreatic polypeptide, respectively.

Endocrine: Tissue which secretes regulatory hormones directly into the bloodstream without the need for an associated duct system.

Enhancer: A nucleic acid sequence that increases the rate of transcription by increasing the activity of a promoter.

Expand: A process by which the number or amount of cells is increased due to cell division. Similarly, the terms “expansion” or “expanded” refers to this process. The terms “proliferate,” “proliferation” or “proliferated” may be used interchangeably with the words “expand,” “expansion,” or “expanded.”

Expressed: Translation of a nucleic acid into a protein. Proteins may be expressed and remain intracellular, become a component of the cell surface membrane, or be secreted into the extracellular matrix or medium.

Exocrine: Secretory tissue which distributes its products, such as enzymes, via an associated duct network. The exocrine pancreas is the part of the pancreas that secretes enzymes required for digestion. The exocrine cells of the pancreas include the centroacinar cells and basophilic cells, which produce secretin and cholecystokinin.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Glucagon: A pancreatic enzyme produced by the pancreatic α cells in vivo. Exemplary glucagon amino acid sequences are shown in GENBANK® accession Nos: NP_002045.1 (pro-protein) (human); NP_032126.1 (mouse), both incorporated by reference. The term “glucagon” also encompasses species variants, homologues, allelic forms, mutant forms, and equivalents thereof, including conservative substitutions, additions, deletions therein not adversely affecting the function, such as binding to the glucagon receptor. Glucagon is encoded by nucleic acid corresponding to GENBANK® Accession No: NM_002054.5 (human); NM_008100.4 (mouse), both incorporated by reference as available on Jan. 4, 2022. The glucagon protein encoded by the glucagon gene, which includes a glucagon promoter in vivo. Glucagon is expressed as a preproprotein that is cleaved into four distinct mature peptides, one of which is glucagon. Glucagon is a pancreatic hormone that counteracts the glucose-lowering action of insulin by stimulating glycogenolysis and gluconeogenesis. Glucagon is a ligand for a specific G-protein linked receptor whose signaling pathway controls cell proliferation.

Heterologous: A heterologous sequence is a sequence that is not normally (in the wild-type sequence) found adjacent to a second sequence. In one embodiment, the sequence is from a different genetic source, such as a virus or organism, than the second sequence. A protein that is heterologous to a glucagon promoter is any protein other than glucagon (it is not glucagon).

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Insulin: A protein hormone involved in the regulation of blood sugar levels that is produced by pancreatic beta cells. In vivo, insulin is produced as a precursor proinsulin, consisting of the B and A chains of insulin linked together via a connecting C-peptide. Insulin itself includes only the B and A chains. Exemplary nucleic acid sequences encoding insulin are provided in GENBANK® Accession No: NM_000207.3 (human) and NM_008386.4 (mouse), as available on Dec. 28, 2021, and are incorporated by reference herein. The term insulin also encompasses species variants, homologues, allelic forms, mutant forms, and equivalents thereof, including conservative substitutions, additions, deletions therein not adversely affecting the structure of function.

Islets of Langerhans: Small discrete clusters of pancreatic endocrine tissue. In vivo, in an adult mammal, the islets of Langerhans are found in the pancreas as discrete clusters (islands) of pancreatic endocrine tissue surrounded by the pancreatic exocrine (or acinar) tissue. In vivo, the islets of Langerhans consist of the α cells, β cells, δ cells, PP cells, and ε cells. Histologically, in rodents, the islets of Langerhans consist of a central core of β cells surrounded by an outer layer of α cells, δ cells, and PP cells. The structure of human islets of Langerhans is different and distinct from rodents. The islets of Langerhans are sometimes referred to herein as “islets.”

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. An isolated cell type has been substantially separated from other cell types, such as a different cell type that occurs in an organ. A purified cell or component can be at least 90%, at least 95%, at least 96%, at least 97%, at least 98%, or at least 99% pure.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes.

Musculoaponeurotic fibrosarcoma oncogene homolog A (MAFA): MAFA is a transcription factor that binds RIPE3b, a conserved enhancer element that regulates pancreatic beta cell-specific expression of the insulin gene (INS; MIM 176730) (Olbrot et al., 2002). MAFA is referred in the art as aliases; v-maf musculoaponeurotic fibrosarcoma oncogene homolog A (avian), hMAFA; RIPE3b1; MAFA. Exemplary MAFA proteins are the MAFA protein of GENBANK® Accession No: NM__194350 (mouse) (SEQ ID NO:3 32 of U.S. Published Patent Application No. 2011/0280842) or NP__963883.2 (Human)(SEQ ID NOs: 33 and 32 of U.S. Published Patent Application No. 2011/0280842); GeneID No: 389692, which are all incorporated by reference. The term MAFA also encompasses species variants, homologues, allelic forms, mutant forms, and equivalents thereof, including conservative substitutions, additions, deletions that do not adversely affecting the structure of function. The term “MAFA”, or “MAFA” protein” as used herein refers to a polypeptide having a naturally occurring amino acid sequence of a MAFA” protein or a fragment, variant, or derivative thereof retains the ability of the naturally occurring protein to bind to DNA and activate gene transcription of Glut2 and pyruvate carboxylase, and other genes such as Glut2, PDX-1, Nkx6.1, GLP-1 receptor, prohormone convertase-1/3 as disclosed in Wang et al., Diabetologia. 2007 February; 50(2): 348-358, which is incorporated herein by reference. Exemplary MAFA nucleic acids are GENBANK® Accession No: NM__201589 (human) (SEQ ID NO:36 32 of U.S. Published Patent Application No. 2011/0280842) and GENBANK® Accession No: NM__194350 (mouse) (SEQ ID NO: 39 32 of U.S. Published Patent Application No. 2011/0280842), which are all incorporated by reference. In addition to naturally-occurring allelic variants of the MAFA sequences that may exist in the population, it will be appreciated that, as is the case for virtually all proteins, a variety of changes can be introduced into the sequences of SEQ ID NO: 3 32 of U.S. Published Patent Application No. 2011/0280842 or SEQ ID NO: 33 32 of U.S. Published Patent Application No. 2011/0280842 (referred to as “wild type” sequences) without substantially altering the functional (biological) activity of the polypeptides. Such variants are included within the scope of the terms “MAFA”, “MAFA protein”, etc. U.S. Published Patent Application No. 2011/0280842 and all of the GENBANK entries are incorporated herein by reference.

Mammal: This term includes both human and non-human mammals. Similarly, the term “subject” includes both human and veterinary subjects.

Neurogenin (Ngn)3: Neurogenin-3 (also known as NEUROG3) is expressed in endocrine progenitor cells and is required for endocrine cell development in the pancreas and intestine. It belongs to a family of basic helix-loop-helix transcription factors involved in the determination of neural precursor cells in the neuroectoderm. Ngn3 is referred in the art as aliases; Neurogenin 3; Atoh5; Math4B; bHLHa7; NEUROG3. Exemplary Ngn3 proteins are provided in GENBANK® Accession No: NM__009719 (mouse) and SEQ ID NO:2 of U.S. Published Patent Application No. 2011/0280842, both incorporated by reference herein or GENBANK® Accession No: NP__033849.3 (Human) and SEQ ID NO: 32 of U.S. Published Patent Application No. 2011/0280842, both incorporated by reference herein; GeneID No: 50674. The term Ngn3 also encompasses species variants, homologues, allelic forms, mutant forms, and equivalents thereof, including conservative substitutions, additions, deletions therein not adversely affecting the structure of function. Human Ngn3 is encoded by nucleic acid corresponding to GENBANK® Accession No: NM__020999 (human), SEQ ID NO:35 of U.S. Published Patent Application No. 2011/0280842 or NM__009719 (mouse), SEQ ID NO: 38 of U.S. Published Patent Application No. 2011/0280842. U.S. Published Patent Application No. 2011/0280842 and these GENBANK® Accession Nos. are incorporated by reference herein. The term “Ngn3”, or “Ngn3 protein” as used herein refers to a polypeptide having a naturally occurring amino acid sequence of a Ngn3 protein or a fragment, variant, or derivative thereof that retains the ability of the naturally occurring protein to bind to DNA and activate gene transcription of NeuroD, Delta-like 1 (Dll1), HeyL, insulinoma-assiciated-1 (IA1), Nk2.2, Notch, HesS, Isl1, Somatostatin receptor 2 (Sstr2) and other genes as disclosed in Serafimidis et al., Stem cells; 2008; 26; 3-16, which is incorporated herein in its entirety by reference. In addition to naturally-occurring allelic variants of the Ngn3 sequences that may exist in the population, it will be appreciated that, as is the case for virtually all proteins, a variety of changes can be introduced into a wild-type sequence (listed above in GENBANK® enteries) without substantially altering the functional (biological) activity of the polypeptides. Such variants are included within the scope of the terms “Ngn3”, “Ngn3 protein”, etc.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see for example, Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, such as version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF (open reading frame): A series of nucleotide triplets (codons) coding for amino acids without any termination codons. These sequences are usually translatable into a peptide.

Pancreatic endocrine cell: An endocrine cell of pancreatic origin that produces one or more pancreatic hormone, such as insulin, glucagon, somatostatin, or pancreatic polypeptide. Subsets of pancreatic endocrine cells include the α (glucagon producing), β (insulin producing) δ (somatostatin producing) or PP (pancreatic polypeptide producing) cells. Additional subsets produce more than one pancreatic hormone, such as, but not limited to, a cell that produces both insulin and glucagon, or a cell that produces insulin, glucagon, and somatostatin, or a cell that produces insulin and somatostatin.

Pancreas duodenal homeobox protein (PDX)1: PDX1 protein is a transcriptional activator of several genes, including insulin, somatostatin, glucokinase, islet amyloid polypeptide, and glucose transporter type 2 (GLUT2). PDX1 is a nuclear protein is involved in the early development of the pancreas and plays a major role in glucose-dependent regulation of insulin gene expression. Defects in the gene encoding the PDX1 protein are a cause of pancreatic agenesis, which can lead to early-onset insulin-dependent diabetes mellitus (NIDDM), as well as maturity onset diabetes of the young type 4 (MODY4). PDX1 is referred in the art as aliases; pancreatic and duodenal homeobox 1, IDX-1, STF-1, PDX-1, MODY4, Ipf1. Exemplary PDX1 proteins are shown in GENBANK® Accession No. NM__008814 (mouse) (SEQ ID NO:1 of U.S. Published Patent Application No. 2011/0280842) or GENBANK® Accession No. NP__000200.1 (Human)(SEQ ID NO: 31 of U.S. Published Patent Application No. 2011/0280842), or Gene ID: 3651, which are all incorporated herein by reference. The term PDX1 also encompasses species variants, homologues, allelic forms, mutant forms, and equivalents thereof, including conservative substitutions, additions, deletions therein not adversely affecting the structure of function. Exemplary nucleic acid sequences are shown in GENBANK® Accession No NM__000209 (human) (SEQ ID NO:34 of U.S. Published Patent Application No. 2011/0280842) or GENBANK® Accession No NM__008814 (mouse)(SEQ ID NO: 37 of U.S. Published Patent Application No. 2011/0280842), which are all incorporated by reference. The term “PDX1”, or “PDX1 protein” as used herein refers to a polypeptide having a naturally occurring amino acid sequence of a PDX1 protein or a fragment, variant, or derivative thereof that at least in part retains the ability of the naturally occurring protein to bind to DNA and activate gene transcription of insulin, somatostatin, glucokinase, islet amyloid polypeptide, and glucose transporter type 2 (GLUT2). In addition to naturally-occurring allelic variants of the PDX1 sequences that may exist in the population, it will be appreciated that, as is the case for virtually all proteins, a variety of changes can be introduced into a wild type sequence (see the listed GENBANK® enteries) without substantially altering the functional (biological) activity of the polypeptides. Such variants are included within the scope of the terms “PDX1”, “PDX1 protein”, etc. The listed GENBANK® Accession Nos. and of U.S. Published Patent Application No. 2011/0280842 are incorporated by reference herein.

Pharmaceutically acceptable carriers: The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or a composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Pre-diabetes: A state in which some, but not all, of the criteria for diabetes are met. For example, a subject can have impaired fasting glycaemia or impaired fasting glucose (IFG). Subjects with fasting glucose levels from 110 to 125 mg/dl (6.1 to 6.9 mmol/l) are considered to have impaired fasting glucose. Subjects with plasma glucose at or above 140 mg/dL (7.8 mmol/L), but not over 200 mg/dL (11.1 mmol/L), two hours after a 75 g oral glucose load are considered to have impaired glucose tolerance.

Predisposition for diabetes: A subject that is at high risk for developing diabetes. A number of risk factors are known to those of skill in the art and include: genetic factors (e.g., carrying alleles that result in a higher occurrence of diabetes than in the average population or having parents or siblings with diabetes); overweight (e.g., body mass index (BMI) greater or equal to 25 kg/m.sup.2); habitual physical inactivity, race/ethnicity (e.g., African-American, Hispanic-American, Native Americans, Asian-Americans, Pacific Islanders); previously identified impaired fasting glucose or impaired glucose tolerance, hypertension (e.g., greater or equal to 140/90 mmHg in adults); HDL cholesterol greater or equal to 35 mg/dl; triglyceride levels greater or equal to 250 mg/dl; a history of gestational diabetes or delivery of a baby over nine pounds; and/or polycystic ovary syndrome. See, e.g., “Report of the Expert Committee on the Diagnosis and Classification of Diabetes Mellitus” and “Screening for Diabetes” Diabetes Care 25(1): S5-S24 (2002).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions replace one amino acid with another amino acid that is similar in size, hydrophobicity, etc. Variations in the cDNA sequence that result in amino acid changes, whether conservative or not, should be minimized in order to preserve the functional and immunologic identity of the encoded protein. The immunologic identity of the protein may be assessed by determining if it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may, for example, be 80, 90 or even 95% or 98% identical to the native amino acid sequence.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Preventing, treating or ameliorating a disease: “Preventing” a disease (such as type 1 diabetes) refers to inhibiting the full development of a disease in a subject with a pre-disposition to develop the disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter can optionally include distal enhancer or repressor elements which can be located as much as several thousand base pairs from the start site of transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987).

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, such as by genetic engineering techniques. A recombinant nucleic acid can include a nucleic acid that has a non-coding function (such as a promoter, origin of replication, ribosome-binding site, etc.). A recombinant protein is one encoded for by a recombinant nucleic acid molecule. In addition, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule (such as a recombinant nucleic acid molecule comprising a glucagon promoter and encoding PDX1 and MAFA) has been packaged. A host cell that comprises the recombinant nucleic acid is referred to as a “recombinant host cell.”

Selectable Marker: A gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select specific cells of interest. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. The term “selectable marker” as used herein can refer to a gene or to an expression product of the gene, e.g., an encoded protein.

Sequence identity of amino acid sequences: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of proteins, such as MAFA or PDX1 are typically characterized by possession of at least about 75%, for example at least about 80%, 90%, 95%, 96%, 97%, 98% or 99% sequence identity counted over the full length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Specific binding agent: An agent that binds substantially only to a defined target. Thus, a β cell specific binding agent is an agent that binds substantially to a β cell, and a pancreatic endocrine cell specific binding agent is an agent that binds substantially only to pancreatic endocrine cells or a subset thereof (and not to pancreatic exocrine cells). Similarly, a pancreatic exocrine cell specific binding agent is an agent that binds substantially to exocrine cells. In one embodiment, the specific binding agent is a monoclonal or polyclonal antibody that specifically binds a type of pancreatic cell.

The term “specifically binds” refers, with respect to a cell, such as a pancreatic endocrine cell, to the preferential association of an antibody or other ligand, in whole or part, with a cell or tissue bearing that antigen and not to cells or tissues lacking that antigen. It is, of course, recognized that a certain degree of non-specific interaction may occur between a molecule and a non-target cell or tissue. Nevertheless, specific binding may be distinguished as mediated through specific recognition of the antigen. Although selectively reactive antibodies bind antigen, they may do so with low affinity. On the other hand, specific binding results in a much stronger association between the antibody (or other ligand) and cells bearing the antigen than between the bound antibody (or other ligand) and cells lacking the antigen. Specific binding typically results in greater than 2-fold, such as greater than 5-fold, greater than 10-fold, or greater than 100-fold increase in amount of bound antibody or other ligand (per unit time) to a cell or tissue expressing the target epitope as compared to a cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Subject: Any mammal, such as humans, non-human primates, pigs, sheep, cows, rodents and the like which is to be the recipient of the particular treatment. In two non-limiting examples, a subject is a human subject or a murine subject.

Therapeutic agent: Used in a generic sense, it includes treating agents, prophylactic agents, and replacement agents. A therapeutic agent can be a nucleic acid molecule encoding MAFA and PDX-1, or a vector encoding these factors.

Therapeutically effective amount: A quantity of a specified pharmaceutical or therapeutic agent (e.g., a recombinant AAV) sufficient to achieve a desired effect in a subject, or in a cell, being treated with the agent, such as increasing insulin production. The effective amount of the agent will be dependent on several factors, including, but not limited to the subject or cells being treated, and the manner of administration of the therapeutic composition.

Transdifferentiate: A process wherein a differentiated cell of one type, such as an alpha cell, differentiates into a differentiated cell of another type, such as a beta cells.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses {Wolff, J. A., ed, Gene Therapeutics, Birkhauser, Boston, USA (1994)}. In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an artificial islet produced by the methods described herein.

Genetic modification of the target cell is an indicium of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like. In some embodiments herein, the vector is an AAV vector.

Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

### Overview of Several Embodiments

Glucagon promoters are disclosed herein, wherein the glucagon promoter consists essentially of, or consists of: a nucleotide sequence at least 95% identical to SEQ ID NO: 1, that functions as a promoter; the nucleotide sequence of SEQ ID NO: 1; a nucleotide sequence at least 95% identical to SEQ ID NO: 2, that functions as a promoter; the nucleotide sequence of SEQ ID NO: 2; a nucleotide sequence at least 95% identical to SEQ ID NO: 3, that functions as a promoter; or the nucleotide sequence of SEQ ID NO: 3. In some embodiments, the glucagon promoter consists of the nucleotide sequence at least 95% identical to SEQ ID NO: 1, that functions as a promoter; the nucleotide sequence of SEQ ID NO: 1; the nucleotide sequence at least 95% identical to SEQ ID NO: 2, that functions as a promoter; the nucleotide sequence of SEQ ID NO: 2; the nucleotide sequence at least 95% identical to SEQ ID NO: 3, that functions as a promoter; or the nucleotide sequence of SEQ ID NO: 3. In other non-limiting examples, the promoter consists of the nucleotide sequence of SEQ ID NO: 1; the nucleotide sequence of SEQ ID NO: 2; or the nucleotide sequence of SEQ ID NO: 3. In a further embodiment, a recombinant nucleic acid molecule including the glucagon promoter operably linked to a nucleic acid molecule encoding at least one heterologous protein is disclosed. The heterologous protein can be Pancreas duodenal homeobox protein (PDX1) and/or Musculoaponeurotic fibrosarcoma oncogene homolog A (MAFA).

In some embodiments, a vector including the recombinant nucleic acid molecule are disclosed. The vector can be a viral vector. The viral vector can be a lentivirus vector, an adenovirus vector or an adeno-associated virus (AAV) vector, such as, but not limited to, an AAV6 vector.

In further embodiments, a host cell is disclosed that is transformed with the recombinant nucleic acid molecule or vector. The host cell can be a mammalian host cell, such as a human host cell or a murine host cell. The host cell can be an alpha cell.

Also disclosed is a method of producing a protein in a host cell. The method includes transforming the host cell with an effective amount of the vector, thereby producing the protein in the host cell. The host cell can be a mammalian host cell, such as a human host cell or a murine host cell. The host cell can be an alpha cell.

In some embodiments, compositions are disclosed that include comprising an effective amount of recombinant nucleic acid molecule or the vector of and a pharmaceutically acceptable carrier. In these embodiments, the heterologous protein can be PDX1 and/or MAFA. The composition can also include a contrast dye for endoscopic retrograde cholangiopancreatography. The contrast dye can be a low-osmolar low-viscosity non-ionic dye, a low-viscosity high-osmolar dye, or a dissociable high-viscosity dye. The contrast dye can be Iopromid, Ioglicinate, or Ioxaglinate.

In further embodiments, methods are disclosed for producing pancreatic beta cells in a subject. These methods include administering to the subject a disclosed vector, wherein the glucagon promoter is operably linked to a nucleic acid molecule encoding PDX1 and a nucleic acid molecule encoding MAFA. In some examples, the vector does not encode Neurogenin 3 (Ngn3) and the subject is not administered any other nucleic acid encoding Ngn3. The vector is administered intraductally into a pancreatic duct of the subject, thereby inducing alpha cells to transdifferentiate into pancreatic beta cells in the subject. In some non-limiting examples, the nucleic acid sequence encoding PDX1 and the nucleic acid sequence encoding MAFA are linked using a connector, such as, but not limited to, a 2A connector. The intraductal administration can include the use of endoscopic retrograde cholangiopancreatography (ERCP). In some embodiments, the subject is not administered an immunosuppressive agent. In more embodiments, the subject is a human. In further embodiments, the subject has type I diabetes.

### Glucagon Promoters, Recombinant Nucleic Acid Molecules, and Vectors

Disclosed herein are glucagon promoters that include a portion of the full-length glucagon promoter.

An exemplary human glucagon promoter
consists of, or consists essentially
of: SEQ ID NO: 1.
Human glucagon promoter 592bp
(SEQ ID NO: 1)
GGGGTTTAATCAATATCCTTAAATTTTCCACAAACATAACATAAATAAAC
TCCACGTTGTGAGGAAGAGAGGATTTTTAATACATATGTGTTGAATGAAT
GATCATTATTTAGATAAATGAATGACTGAAGTGATTGTTATATTCAGGTA
AATTCATCATGGCTAGGTAGCAAACCAAAGACTTGTAAGAACCTCAAATG
AGGACATGCACAAAACAGGGATGGCCATGGGCTACGTAATTTCAAGGTCT
TTTGTCTTCAACGTCAAAATTCACTTTAGAGAACTTAAGTGATTTTCATG
CGTGATTGAAAGTAGAAGGTGGATTTCCAAGCTGCTCTCTCCATTCCCAA
CCAAAAAAAAAAAAAAAAGATACAAGAGTGCATAAAAAGTTTCCAGGTCT
CTAAGGTCTCTCACCCAATATAAGCATAGAATGCAGATGAGCAAAGTGAG
TGGGAGAGGGAAGTCATTTGTAACAAAAACTCATTATTTACAGATGAGAA
ATTTATATTGTCAGCGTAATATCTGTGAGGCTAAACAGAGCTGGAGAGTA
TATAAAAGCAGTGCGCCTTGGTGCAGAAGTACAGAGCTTAGG
Another exemplary glucan promoter
consists of, or consists essentially
of, SEQ ID NO: 2.
Human glucagon promoter 660bp
(SEQ ID NO: 2)
TGGATGTTTAATCTTCATTTTGCTCCATCCTTTCTGCCTGAATTCCATTT
ATTAAAACAGAACACATAGGGGTTTAATCAATATCCTTAAATTTTCCACA
AACATAACATAAATAAACTCCACGTTGTGAGGAAGAGAGGATTTTTAATA
CATATGTGTTGAATGAATGATCATTATTTAGATAAATGAATGACTGAAGT
GATTGTTATATTCAGGTAAATTCATCATGGCTAGGTAGCAAACCAAAGAC
TTGTAAGAACCTCAAATGAGGACATGCACAAAACAGGGATGGCCATGGGC
TACGTAATTTCAAGGTCTTTTGTCTTCAACGTCAAAATTCACTTTAGAGA
ACTTAAGTGATTTTCATGCGTGATTGAAAGTAGAAGGTGGATTTCCAAGC
TGCTCTCTCCATTCCCAACCAAAAAAAAAAAAAAAAGATACAAGAGTGCA
TAAAAAGTTTCCAGGTCTCTAAGGTCTCTCACCCAATATAAGCATAGAAT
GCAGATGAGCAAAGTGAGTGGGAGAGGGAAGTCATTTGTAACAAAAACTC
ATTATTTACAGATGAGAAATTTATATTGTCAGCGTAATATCTGTGAGGCT
AAACAGAGCTGGAGAGTATATAAAAGCAGTGCGCCTTGGTGCAGAAGTAC
AGAGCTTAGG
A further exemplary glucagon promoter
consists of, or consists essentially
of, SEQ ID NO: 3.
Mouse glucagon promoter 515bp
(SEQ ID NO: 3)
TTGACATTTAAAATCTCCCCACAAAGAGAATACAAATTTATTCCATACAA
TGTGGATGAGTGGGTTATTGATATGTGTGTACTGGGTGAGTGATTTGTTT
GCCTAGATGAATGATCTATCACTTATTCAGGAACTCAGGATATAGCCAAA
TACCAAATCAAGGGATAAGACCCTCAAATGAGACTAGGCTCATTTGACGT
CAAAATTCACTTGAGAGAACTTTAGCAGTTTTTCGTGCCTGACTGAGACC
GAAGGGTGGATCTCCAAACTGCCCTTTCCATTCCCAAACAGAAAGGCACA
AGAGTAAATAAAATGTTTCCGGGCCTCTGCGGTCTCAACCCGGTATCAGC
GTAAAAAGCAGATGAGCAAAGTGAGTGGGCGAGTGAAATCATTTGAACAA
AACCCCATTATTTACAGATGAGAAATTTATATTGTCAGCGTAATATCTGC
AAGGCTAAACAGCCTGGAGAGCATATAAAAGCACAGCACCCTGGTGCAGA
AGGGCAGAGCTTGGG

One of skill in the art will readily appreciate that variants of these promoters are of use, such as promoters at least 95%, 96%, 97%, 98%, 99% identical to one of SEQ ID NO: 1, SEQ ID NO: 2, or SEQ ID NO: 3, provided the promoter functions, such that a heterologous nucleic acid operably linked to the promoter is expressed when transferred into a host cell that expresses glucagon. In additional embodiments, the promoter can include at most 15, 14, 13, 12, 11, 10, 9, 8, 7, 6, 5, 4, 3, 2, 1 nucleic acid substitutions in any one of SEQ ID NOs: 1-3, provided the promoter functions, such that a heterologous nucleic acid operably linked to the promoter can be expressed when transferred into a host cell.

In some embodiments, the variants of the promoter of SEQ ID NO: 1 are 592 base pairs in length. In some non-limiting examples, the promoters is at least 95%, 96%, 97%, 98%, 99% identical to SEQ ID NO: 1 and is 592 nucleotides in length, and the promoter functions, such that a heterologous nucleic acid operably linked to the promoter is expressed when transferred into a host cell that expresses glucagon. In additional embodiments, the promoter includes at most 15, 14, 13, 12, 11, 10, 9, 8, 7, 6, 5, 4, 3, 2, 1 nucleic acid substitutions in SEQ ID NO: 1, and is 592 nucleotides in length, and the promoter functions, such that a heterologous nucleic acid operably linked to the promoter can be expressed when transferred into a host cell.

In other embodiments, the variants of the promoter of SEQ ID NO: 2 are 660 base pairs in length. In some embodiments, the variants of the promoter of SEQ ID NO: 2 are 660 base pairs in length. In some non-limiting examples, the promoters is at least 95%, 96%, 97%, 98%, 99% identical to SEQ ID NO: 2 and is 660 nucleotides in length, and the promoter functions, such that a heterologous nucleic acid operably linked to the promoter is expressed when transferred into a host cell that expresses glucagon. In additional embodiments, the promoter includes at most 15, 14, 13, 12, 11, 10, 9, 8, 7, 6, 5, 4, 3, 2, 1 nucleic acid substitutions in SEQ ID NO: 2, and is 660 nucleotides in length, and the promoter functions, such that a heterologous nucleic acid operably linked to the promoter can be expressed when transferred into a host cell.

In further embodiments, the variants of the promoter of SEQ ID NO: 3 are 515 base pairs in length. In some non-limiting examples, the promoters is at least 95%, 96%, 97%, 98%, 99% identical to SEQ ID NO: 3 and is 515 nucleotides in length, and the promoter functions, such that a heterologous nucleic acid operably linked to the promoter is expressed when transferred into a host cell that expresses glucagon. In additional embodiments, the promoter includes at most 15, 14, 13, 12, 11, 10, 9, 8, 7, 6, 5, 4, 3, 2, 1 nucleic acid substitutions in SEQ ID NO: 3, and is 515 nucleotides in length, and the promoter functions, such that a heterologous nucleic acid operably linked to the promoter can be expressed when transferred into a host cell.

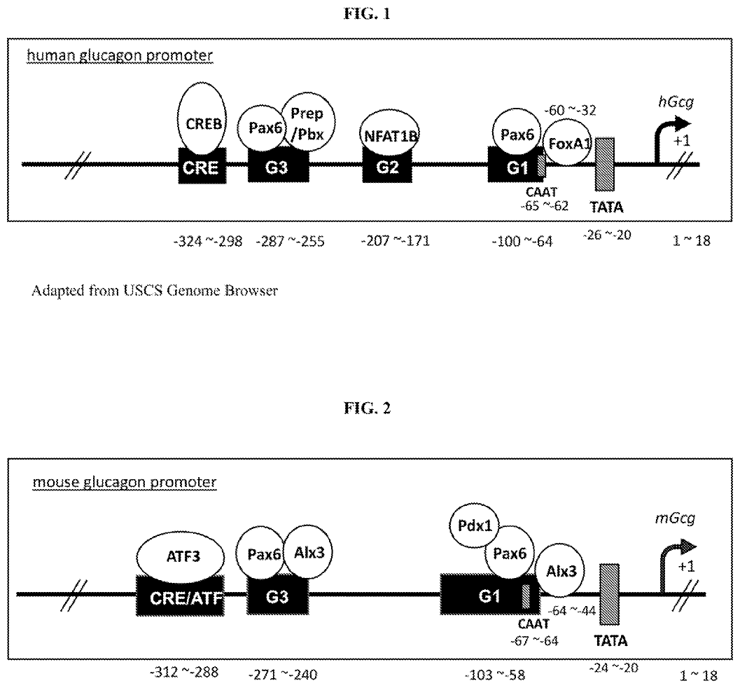

In further embodiments, the variations in the promoter sequence are outside the regions shown in FIG. 1 and FIG. 2. Thus, in some embodiments, the promoter includes the CRE, G3, G2 and/or G1 domain of SEQ ID NOs: 1 and 2, as illustrated in FIG. 1. In other embodiments, the promoter includes the CRE/ATF, G3 and G1 domains of SEQ ID NO: 3.

In SEQ ID NOs: 1 and 2, the G1 domain nucleotide sequence is: ACAAAAACTCATTATTTACAGATGAGAAATTTATATTG (SEQ ID NO: 12); the G2 domain nucleotide sequence is: CAAGAGTGCATAAAAAGTTTCCA (SEQ ID NO: 13): the G3 domain nucleotide sequence is: AGTGATTTTCATGCGTGATTGAAAGTAGAAGGT (SEQ ID NO: 14); the CRE site nucleotide sequence is: TTTGTCTTCAACGTCAAAATTCACTTT (SEQ ID NO: 15). In some embodiments, the promoter is at least 95%, 96%, 97%, 98%, 99% identical to one of SEQ ID NO: 1 or SEQ ID NO: 2, and includes the nucleic acid sequences of SEQ ID NOs: 12-15. In some non-limiting examples, the promoter is at least 95%, 96%, 97%, 98%, 99% identical to SEQ ID NO: 1, includes the nucleic acid sequences of SEQ ID NOs: 12-15, and is 592 nucleotides in length. In other non-limiting examples, the promoter is at least 95%, 96%, 97%, 98%, 99% identical to SEQ ID NO: 2, includes the nucleic acid sequences of SEQ ID NOs: 12-15, and is 660 nucleotides in length. In additional embodiments, the promoter can include at most 15, 14, 13, 12, 11, 10, 9, 8, 7, 6, 5, 4, 3, 2, 1 nucleic acid substitutions in SEQ ID NO: 1 or SEQ ID NO: 2, and includes the nucleic acid sequences of SEQ ID NOs: 12-15. In these embodiments, the promoter functions, such that a heterologous nucleic acid operably linked to the promoter is expressed when transferred into a host cell that expresses glucagon.

In SEQ ID NO: 3, the G1 region nucleotide sequence is: GAACAAAACCCCATTATTTACAGATGAGAAATTTATATTGTCAGCG (SEQ ID NO: 16); the (G3 region nucleotide sequence: AGTTTTTCGTGCCTGACTGAGACCGAAGGGTG (SEQ ID NO: 17); and the CRE/ATF domain sequence is: AGGCTCATTTGACGTCAAAATTCAC (SEQ ID NO: 18).

In some embodiments, the promoter is at least 95%, 96%, 97%, 98%, 99% identical SEQ ID NO: 3, and includes the nucleic acid sequences of SEQ ID NOs: 16-18. In some non-limiting examples, the promoter is at least 95%, 96%, 97%, 98%, 99% identical to SEQ ID NO: 3, includes the nucleic acid sequences of SEQ ID NOs: 16-18, and is 515 nucleotides in length. In additional embodiments, the promoter can include at most 15, 14, 13, 12, 11, 10, 9, 8, 7, 6, 5, 4, 3, 2, 1 nucleic acid substitutions in SEQ ID NO: 3, and includes the nucleic acid sequences of SEQ ID NOs: 16-18. In these embodiments, the promoter functions, such that a heterologous nucleic acid operably linked to the promoter is expressed when transferred into a host cell that expresses glucagon.

Additional nucleotides can be added, provided the promoter functions, such that a heterologous nucleic acid operably linked to the promoter is expressed when transferred into a host cell. The promoter can include the nucleic acid sequence set forth as SEQ ID NO: 1 or SEQ ID NO: 2, and an additional 10, 20, 30, 40, 50, 60, 70, 80, 90, or 100 nucleotides of the full-length human glucagon promoter. The promoter can include the nucleic acid sequence set forth as SEQ ID NO: 3, and an additional 10, 20, 30, 40, 50, 60, 70, 80, 90, or 100 nucleotides of the full-length mouse glucagon promoter, provided the promoter functions to provide transcription of a nucleic acid encoding a heterologous protein.

In some embodiments, another nucleic acid sequence can be linked to the promoter, such as an enhancer. The promoter can be linked to a Kozak sequence. The promoter can be linked to an intron between a promoter and a Kozak sequence, wherein the intron works as an enhancer. The

In other embodiments, disclosed promoters can be operably linked to a heterologous nucleic acid molecule, such as a nucleic acid encoding a heterologous protein. In some embodiments, the heterologous nucleic acid encodes PDX1 or MAFA. In further embodiments, the heterologous nucleic acid encodes PDX1 and MAFA. In yet other embodiments, the heterologous nucleic acid encodes PDX1 and/or MAFA and does not encode Ngn3. The heterologous nucleic acid can encode a selectable marker, which includes, but are not limited to, a protein whose expression can be readily detected such as a fluorescent or luminescent protein or an enzyme that acts on a substrate to produce a colored, fluorescent, or luminescent substance (“detectable markers”). There are other nucleic acid molecule of use, such as a nucleic acid molecule that encodes drug resistance or provides a function that can be used to purify cells. Selectable markers include neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also selectable makers.

In some embodiments, the promoter functions such that MAFA and/or PDX1 transcripts are produced. In specific non-limiting examples, the promoter is operably linked to a nucleic acid encoding MAFA and a nucleic acid encoding PDX1, but is not operably linked to a nucleic acid encoding Ngn3. U.S. Published Patent Application No. 2011/0280842, incorporated by reference herein, provides PDX1, MAFA and Ngn3 amino acid and nucleic acid sequences.

In some embodiments, the promoter is operably linked to a heterologous nucleic acid molecule encoding MAFA, such as the amino acid sequence set forth as: MAAELAMGAE LPSSPLAIEY VNDFDLMKFE VKKEPPEAER FCHRLPPGSL SSTPLSTPCS SVPSSPSFCA PSPGTGGGGG AGGGGGSSQA GGAPGPPSGG PGAVGGTSGK PALEDLYWMSGYQHHLNPEA LNLTPEDAVE ALIGSGHHGA HHGAHHPAAA AAYEAFRGPG FAGGGGADDMGAGHHHGAHH AAHHHHAAHH HHHHHHHHGG AGHGGGAGHH VRLEERFSDD QLVSMSVRELNRQLRGFSKE EVIRLKQKRR TLKNRGYAQS CRFKRVQQRH ILESEKCQLQ SQVEQLKLEVGRLAKERDLY KEKYEKLAGR GGPGSAGGAG FPREPSPPQA GPGGAKGTAD FFL (human MAFA, SEQ ID NO: 4, GENBANK® Accession No. NP_963883.2, May 10, 2014, incorporated herein by reference), or MAAELAMGAE LPSSPLAIEY VNDFDLMKFE VKKEPPEAER FCHRLPPGSL SSTPLSTPCSSVPSSPSFCA PSPGTGGGAG GGGSAAQAGG APGPPSGGPG TVGGASGKAV LEDLYWMSGYQHHLNPEALN LTPEDAVEAL IGSGHHGAHH GAHHPAAAAA YEAFRGQSFA GGGGADDMGAGHHHGAHHTA HHHHSAHHHH HHHHHHGGSG HHGGGAGHGG GGAGHHVRLE ERFSDDQLVSMSVRELNRQL RGFSKEEVIR LKQKRRTLKN RGYAQSCRFK RVQQRHILES EKCQLQSQVEQLKLEVGRLA KERDLYKEKY EKLAGRGGPG GAGGAGFPRE PSPAQAGPGA AKGAPDFFL (mouse MAFA, SEQ ID NO: 5, GENBANK® Accession No. NP_919331, Apr. 26, 2014, incorporated herein by reference. MAFA is a beta cell specific and glucose regulated transcription factor for insulin gene expression.

A disclosed promoter can be operably linked to a heterologous nucleic acid molecule encoding a MAFA protein that has an amino acid sequence at least 95%, 96%, 97%, 98%, 99% or 100% identical to SEQ ID NO: 4 or SEQ ID NO: 5, wherein the protein functions as a transcription factor. The heterologous nucleic acid molecule can encode a MAFA protein that includes at most 10, 9, 8, 7, 6, 5, 4, 3, 2, or 1 conservative amino acid substitutions in SEQ ID NO: 4 or SEQ ID NO: 5, wherein the protein functions as a transcription factor. The disclosed promoter can be operably linked to a nucleic acid molecule that encodes a protein that has the amino acid sequence of SEQ ID NO: 40 r SEQ ID NO: 5.

In some embodiments, a disclosed promoter can be operably linked to a heterologous nucleic acid molecule encoding a human PDX1 amino acid sequence including the amino acid sequence set forth as:

MNGEEQYYAA TQLYKDPCAF QRGPAPEFSA SPPACLYMGR
QPPPPPPHPF PGALGALEQGSPPDISPYEV PPLADDPAVA
HLHHHLPAQL ALPHPPAGPF PEGAEPGVLE EPNRVQLPFP
WMKSTKAHAW KGQWAGGAYA AEPEENKRTR TAYTRAQLLE
LEKEFLFNKY ISRPRRVELAVMLNLTERHI KIWFQNRRMK
WKKEEDKKRG GGTAVGGGGV AEPEQDCAVT SGEELLALPP
PPPPGGAVPP AAPVAAREGR LPPGLSASPQ PSSVAPRRPQ
EPR

(human PDX1, SEQ ID NO: 6, GENBANK Accession No. NP_000200.1, Mar. 15, 2015, incorporated herein by reference),
or

MNSEEQYYAATQLYKDPCAFQRGPVPEFSANPPACLYMGRQPPPPPPPQF
TSSLGSLEQGSPPDISPYEVPPLASDDPAGAHLHHHLPAQLGLAHPPPGP
FPNGTEPGGLEEPNRVQLPFPWMKSTKAHAWKGQWAGGAYTAEPEENKRT
RTAYTRAQLLELEKEFLFNKYISRPRRVELAVMLNLTERHIKIWFQNRRM
KWKKEEDKKRSSGTPSGGGGGEEPEQDCAVTSGEELLAVPPLPPPGGAVP
PGVPAAVREGLLPSGLSVSPQPSSIAPLRPQEPR

(mouse PDX1, SEQ ID NO: 7, GENBANK Accession No: NM_008814.3, Feb. 15, 2015, incorporated herein by reference). PDX1 is a transcriptional activator of several genes, including insulin, somatostatin, glucokinase, islet amyloid polypeptide, and glucose transporter type 2.

The heterologous nucleic acid molecule can encode a PDX1 protein that has an amino acid sequence at least 95%, 96%, 97%, 98%, 99% or 100% identical to SEQ ID NO: 6 or SEQ ID NO: 7, wherein the protein functions as a transcription factor. The heterologous nucleic acid molecule can encode a PDX1 protein that includes at most 10, 9, 8, 7, 6, 5, 4, 3, 2, or 1 conservative amino acid substitutions in SEQ ID NO: 6 or SEQ ID NO: 7, wherein the protein functions as a transcription factor. The disclosed promoter can be operably linked to a nucleic acid molecule that encodes a protein that has the amino acid sequence of SEQ ID NO: 6 or SEQ ID NO: 7.

The heterologous nucleic acid molecule can encode both MAFA and PDX1. In some embodiments, the heterologous nucleic acid molecules encoding MAFA and PDX1 are separated by a connector. In specific non-limiting examples, the connector is 2A. The nucleic acid sequence of the 2A connector is shown below:

(SEQ ID NO: 8)
CGCGCCAAGCGCGGCTCCGGCGCCACCAACTTCTCCCTGCTGAAGCAG.

Nucleic acid sequences of additional exemplary connectors are:

(SEQ ID NO: 9)
CGCGCCAAGCGCGGCTCCGGCCAGTGCACCAACTACGCCCTGCTGAAGCT
GGCCGGCGACGTGGAGTCCAACCCCGGCCCC;
and
(SEQ ID NO: 10)
CGCGCCAAGCGCGGCTCCGGCGAGGGCAGAGGAAGTCTTCTAACATGCGG
TGACGTGGAGGAGAATCCCGGCCCT.

Suitable connectors also include a nucleic acid sequence with at most 1, 2, 3, 4, or 5 substitutions in one of SEQ ID NO: 8-10. Suitable connectors are, for example, 40 to 90 nucleotides in length, such as 45 to 85 nucleotides in length, such as 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 nucleotides in length. These connectors can be included in a heterologous nucleic acid molecule between a nucleic acid molecule encoding MAFA and a nucleic acid molecule encoding PDX1. The nucleic acid molecule encoding MAFA can be 5′ to the nucleic acid molecule encoding PDX1. The nucleic acid molecule encoding MAFA can be 3′ to the nucleic acid molecule encoding PDX1.

In some embodiments, the heterologous nucleic acid molecule encodes MAFA and PDX1, but does not encode Ngn3, for example, the Ngn3 protein of GENBANK® Accession No: NM__009719 (mouse), Feb. 15, 2015 and GENBANK® Accession No: NP_033849.3 (Human), Feb. 15, 2015. An exemplary Ngn3 protein is shown below:

(SEQ ID NO: 11)

MAPHPLDALT IQVSPETQQP FPGASDHEVL SSNSTPPSPT
LIPRDCSEAE VGDCRGTSRKLRARRGGRNR PKSELALSKQ
RRSRRKKAND RERNRMHNLN SALDALRGVL PTFPDDAKLT
KIETLRFAHN YIWALTQTLR IADHSFYGPE PPVPCGELGS
PGGGSNGDWG SIYSPVSQAGNLSPTASLEE FPGLQVPSSP
SYLLPGALVF SDFL

Thus, in some embodiments, the heterologous nucleic acid molecule does not encode a protein that has an amino acid sequence at least 95%, 96%, 97%, 98%, 99% or 100% identical to SEQ ID NO: 11, wherein the protein functions as a transcription factor. The heterologous nucleic acid molecule does not encode a protein that includes at most 10, 9, 8, 7, 6, 5, 4, 3, 2, or 1 conservative amino acid substitutions in SEQ ID NO: 11, wherein the protein functions as a transcription factor.

However, in some embodiments, the heterologous nucleic acid molecule encodes MAFA and PDX1, and encodes Ngn3, or just encodes Ngn3. Thus, in some embodiments, the heterologous nucleic acid molecule encodes a protein that has an amino acid sequence at least 95%, 96%, 97%, 98%, 99% or 100% identical to SEQ ID NO: 11, wherein the protein functions as a transcription factor. The heterologous nucleic acid molecule can encode a protein that includes at most 10, 9, 8, 7, 6, 5, 4, 3, 2, or 1 conservative amino acid substitutions in SEQ ID NO: 11, wherein the protein functions as a transcription factor. The heterologous nucleic acid molecule can encode a protein with the amino acid sequence of SEQ ID NO: 11.

It may be desirable to include a polyadenylation signal to effect proper termination and polyadenylation of the gene transcript. Exemplary polyadenylation signals have been isolated from beta globin, bovine growth hormone, SV40, and the herpes simplex virus thymidine kinase genes.

Also disclosed are a vector including the promoter and the heterologous nucleic acid. Optionally, other transcription control sequences, such as one or more enhancer elements, which are binding recognition sites for one or more transcription factors that increase transcription above that observed for the promoter alone, can be included in the vector. Procedures for producing vectors and cloning can be found, for example, in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

Numerous viral vectors are known in the art, including polyoma; SV40 (Madzak et al., 1992, J. Gen. Virol., 73:15331536); adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Nad. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256); vaccinia virus (Mackett et al., 1992, Biotechnology, 24:495-499); adeno-associated virus (Muzyczka, 1992, Curr. Top. Microbiol. Immunol., 158:91-123; On et al., 1990, Gene, 89:279-282); herpes viruses, including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199); Sindbis viruses (H. Herweijer et al., 1995, Human Gene Therapy 6:1161-1167; U.S. Pat. Nos. 5,091,309, 5,2217,879); alphaviruses (S. Schlesinger, 1993, Trends Biotechnol. 11:18-22; I. Frolov et al., 1996, Proc. Natl. Acad. Sci. USA 93:11371-11377); and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739).

The vector can be a viral vector. Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors, lentivirus vectors and poliovirus vectors. Specific exemplary vectors are poxvirus vectors, such as vaccinia virus, fowlpox virus and a highly attenuated vaccinia virus (MVA), adenovirus, baculovirus, yeast, and the like. The vector can be a retroviral vector, a lentiviral vector, an adenovirus vector. Adeno-associated virus vectors (AAV) are disclosed in additional detail below, and are of use in the disclosed methods. The AAV vector can be AAV6.

Defective viruses, that entirely or almost entirely lack viral genes, can be used. Use of defective viral vectors allows for administration to specific cells without concern that the vector can infect other cells. In some examples, the vector is an attenuated adenovirus vector, such as the vector described by Stratford-Perricaudet et al. (J. Clin. Invest., 90:626-630 1992; La Salle et al., Science 259:988-990, 1993); and a defective adeno-associated virus vector (Samulski et al., J. Virol., 61:3096-3101, 1987; Samulski et al., J. Virol., 63:3822-3828, 1989; Lebkowski et al., Mol. Cell. Biol., 8:3988-3996, 1988).

In some embodiments, the vector includes a disclosed promoter operably linked to a nucleic acid encoding PDX1 and MAFA. In more embodiments, the vector does not include a nucleic acid encoding Ngn3. The vector can be, for example, a lentivirus vector, an adenovirus vector, or an adeno-associated virus (AAV) vector, such as, but not limited to, and AAV6 vector.

In some embodiments, a vector of use includes a gene encoding a selectable marker, which includes, but are not limited to, a protein whose expression can be readily detected such as a fluorescent or luminescent protein or an enzyme that acts on a substrate to produce a colored, fluorescent, or luminescent substance (“detectable markers”). There are other genes of use, such as genes that encode drug resistance of provide a function that can be used to purify cells. Selectable markers include neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also selectable makers.

The disclosed promoter, operably linked to a heterologous nucleic acid molecule, can be included in adenoviral vectors and/or adeno-associated viral vectors. AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. In some embodiments the AAV includes a disclosed promoter operably linked to a nucleic acid encoding PDX1 and MAFA, but does not include a nucleic acid encoding Ngn3. Further provided are recombinant vectors, such as recombinant adenovirus vectors and recombinant adeno-associated virus vectors comprising a nucleic acid molecule disclosed herein. In some embodiments, the AAV is AAV6. However, the AAV serotype can be any other suitable AAV serotype, such as AAV1, AAV2, AAV3, AAV4, AAV5, AAV6, AAV7, AAV9, AAV10, AAV11 or AAV12, or a hybrid of two or more AAV serotypes (such as, but not limited to AAV2/1, AAV2/7, AAV2/8 or AAV2/9).

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008). In some embodiments, these elements are included in the AAV vector.

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008). In some embodiments, these elements are included in the AAV vector.

AAV vectors can be used for gene therapy. Exemplary AAV of use are AAV2, AAV5, AAV6, AAV8 and AAV9. In some embodiments, the AAV is an AAV6 vector.

Adenovirus, AAV2 and AAV8 are capable of transducing cells in the pancreas. Thus, any of a AAV2 or AAV8 vector can be used in the methods disclosed herein. However, AAV6 and AAV9 vectors are also of use. In one non-limiting example, the AAV vector is an AAV6 vector.

Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of an AAV vector including a disclosed promoter for the methods disclosed herein.

AAV possesses several additional desirable features for a gene therapy vector, including the ability to bind and enter target cells, enter the nucleus, the ability to be expressed in the nucleus for a prolonged period of time, and low toxicity. AAV can be used to transfect cells, and suitable vector are known in the art, see for example, U.S. Published Patent Application No. 2014/0037585, incorporated herein by reference. Methods for producing AAV vectors suitable for gene therapy are well known in the art (see, for example, U.S. Published Patent Application Nos. 2012/0100606; 2012/0135515; 2011/0229971; and 2013/0072548; and Ghosh et al., Gene Ther 13(4):321-329, 2006), and can be utilized with the methods disclosed herein.

AAV8 vectors are disclosed, for example, in U.S. Pat. No. 8,692,332, which is incorporated by reference herein. An exemplary AAV8 nucleic acid sequence is shown in FIG. 1 and SEQ ID NO: 1 of U.S. Pat. No. 8,692,332. It is disclosed that AAV nucleic acid sequence can be greater than about 90%, 95%, 98% or 99% identical to this nucleic acid sequence. The location and sequence of the capsid, rep 68/78, rep 40/52, VP1, VP2 and VP3 are disclosed in this U.S. Pat. No. 8,692,332. The location and hypervariable regions of AAV8 are also provided.

The vectors of use in the methods disclosed herein can contain nucleic acid sequences encoding an intact AAV capsid which may be from a single AAV serotype (e.g., AAV2, AAV, 6, AAV8 or AAV9, such as AAV6). As disclosed in U.S. Pat. No. 8,692,332, vectors of use also can be recombinant, and thus can contain sequences encoding artificial capsids which contain one or more fragments of the AAV6 capsid fused to heterologous AAV or non-AAV capsid proteins (or fragments thereof). These artificial capsid proteins are selected from non-contiguous portions of the AAV2, AAV8 or AAV9 capsid or from capsids of other AAV serotypes. For example, a AAV vector may have a capsid protein comprising one or more of the AAV8 capsid regions selected from the VP2 and/or VP3, or from VP1, or fragments thereof selected from amino acids 1 to 184, amino acids 199 to 259; amino acids 274 to 446; amino acids 603 to 659; amino acids 670 to 706; amino acids 724 to 738 of the AAV8 capsid, see SEQ ID NO: 2 of U.S. Pat. No. 8,692,332. In another example, it may be desirable to alter the start codon of the VP3 protein to GTG. Alternatively, the AAV may contain one or more of the AAV serotype 8 capsid protein hypervariable regions, for example aa 185-198; aa 260-273; aa447-477; aa495-602; aa660-669; and aa707-723 of the AAV8 capsid set forth in SEQ ID NO: 2 of U.S. Pat. No. 8,692,332.

In some embodiments, an AAV is generated having an AAV serotype 6 capsid. To produce the vector, a host cell which can be cultured that contains a nucleic acid sequence encoding an adeno-associated virus (AAV) serotype 6 capsid protein, or fragment thereof, as defined herein; a functional rep gene; a minigene composed of, at a minimum, AAV inverted terminal repeats (ITRs) and a transgene, such as a transgene encoding PDX1 and MAFA; and sufficient helper functions to permit packaging in the AAV6 capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. In some embodiments, a stable host cell will contain the required component(s) under the control of an inducible promoter. However, the required component(s) can be under the control of a constitutive promoter. Examples of suitable inducible and constitutive promoters are provided below. Similar methods can be used to generate a AAV2, AAV8 or AAV9 vector and/or virions.

In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing a AAV can be delivered to the packaging host cell in the form of any genetic element which transfer the sequences carried thereon. The selected genetic element may be delivered by any suitable method, including those described herein. The methods used to construct vectors are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating AAV virions are well known and the selection of a suitable method is not a limitation on the present invention. See, e.g., K. Fisher et al, J. Virol., 70:520-532 (1993) and U.S. Pat. No. 5,478,745. In some embodiments, selected AAV components can be readily isolated using techniques available to those of skill in the art from an AAV serotype, including AAV8. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GENBANK®.

In some embodiments, the adenovirus and AAV vectors disclosed herein include a disclosed promoter operably linked to a nucleic acid encoding PDX1 and MAFA. In some embodiments, the adenovirus and AAV vectors do not include a nucleic acid encoding Ngn3.

In some embodiments, the promoter consists essentially of, or consists of, one of SEQ ID NOs: 1-3. Thus, in specific examples, the heterologous nucleic acid encodes PDX and/or MAFA. In additional examples, the heterologous nucleic acid encodes PDX1 and/or MAFA and does not encode Ngn3. In some embodiments, the promoter functions such that both MAFA and/or PDX1 transcripts are produced. In specific non-limiting examples, the promoter is operably linked to a nucleic acid encoding MAFA and a nucleic acid encoding PDX1 but is not operably linked to a nucleic acid encoding NGN3. In other embodiments, a vector including a disclosed promoter and the heterologous nucleic acid, such as an AAV vector, for example and AAV6 vector, does not include a nucleic acid encoding Ngn3.

In some embodiments, host cells can be produced that are transformed with these recombinant nucleic acid molecules. These host cells can be mammalian host cells, such as mouse and human host cells. The host cells can be alpha cells. Methods are disclosed for producing a protein in a host cell, that include transforming the host cell with an effective amount of the vector, thereby producing the protein in the host cell.

The method can include isolating the protein. The heterologous protein can be isolated and purified by standard methods including, but not limited to, chromatography (e.g., ion exchange, affinity, size exclusion, and hydroxyapatite chromatography), gel filtration, centrifugation, or differential solubility, ethanol precipitation, immunoaffinity purification, or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference).

For immunoaffinity chromatography, the protein can be isolated by binding it to an affinity column comprising a specific binding agent, such as antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin can be added at any or all stages in order to reduce or eliminate degradation of the polypeptide or protein during the purification process. Protease inhibitors can be used when cells must be lysed in order to isolate and purify the expressed polypeptide or protein. One of ordinary skill in the art will appreciate that the exact purification technique will vary depending on the character of the protein to be purified, the type of the cells from which the polypeptide or protein is expressed, and the composition of the medium in which the cells were grown.

### Pharmaceutical Compositions and Administration to the Pancreatic Duct

Methods are provided for producing pancreatic beta cells in a subject. The disclosed glucagon promoters provide a high level of expression in alpha cells, providing increased transdifferentiation to beta cells. In some embodiments, MAFA examples and PDX1 expression can be increased in alpha cells, providing increased transdifferentiation into beta cells. These methods include administering to the subject a vector, such as an adenovirus vector or an AAV vector, encoding heterologous PDX1 and MAFA. In some embodiments, vector does not include a nucleic acid encoding Ngn3. In further embodiments, the subject is not administered any other nucleic acid encoding Ngn3. Exemplary methods are disclosed, for example, in U.S. Pat. No. 10,071,172, incorporated herein by reference.

For in vivo delivery, a vector, such as an adenovirus or an AAV vector can be formulated into a pharmaceutical composition and will generally be administered locally or systemically. In some embodiments, the vector is administered directly to the pancreas. In other embodiments, intraductally into a pancreatic duct of the subject. In other embodiments, the subject has diabetes, such as type 1 diabetes.

In some embodiments, methods are provided for producing pancreatic beta cells from pancreatic alpha cells in a subject. These methods include administering to the subject a vector comprising a disclosed promoter operably linked to a nucleic acid molecule encoding PDX1 and MAFA. In some embodiments, the vector does not encode Ngn3 and the subject is not administered any other nucleic acid encoding Ngn3. In more embodiments, the vector is administered intraductally into a pancreatic duct of the subject.

In additional embodiments, methods are provided for treating diabetes type 1 or pre-diabetes in a subject. The subject can be any mammalian subject, including human and veterinary subjects. The subject can be a child or an adult. The method can include selecting a subject of interest, such as a subject with diabetes. The subject can also be administered insulin. The method can include measuring beta cell number.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dL (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes Care 33:S11-61, 2010, incorporated herein by reference).

The disclosed methods produce pancreatic beta cells in a subject. Generally, these cells produce insulin. In some embodiments, the subject is a subject with type 1 diabetes and the pancreatic beta cells produced by the disclosed methods are not recognized by the immune system of the subject. In some embodiments, T cell and/or B cells do not produce an immune response to the pancreatic beta cells produced by the disclosed methods. Thus, in some embodiments, the subject does not mount an autoimmune response to the pancreatic beta cells produced by the disclosed methods. In specific non-limiting examples, the subject does not have immune destruction of the pancreatic beta cells, and does not exhibit an increased lymphocyte invasion of the islets. In some embodiments, the disclosed method transdifferentiate alpha cells into beta cells. Appropriate doses of a disclosed vector depend on the subject being treated (e.g., human or nonhuman primate or other mammal), age and general condition of the subject to be treated, the severity of the condition being treated, the mode of administration of the AAV vector/virion, among other factors. An appropriate effective amount can be readily determined by one of skill in the art. Thus, a “therapeutically effective amount” will fall in a relatively broad range that can be determined through clinical trials. The method can include measuring an outcome, such as insulin production, improvement in a fasting plasma glucose tolerance test, or pancreatic beta cell number. The method can include administering other therapeutic agents, such as insulin. The method can also include having the subject make lifestyle modifications.

For example, for in vivo injection, a therapeutically effective dose can be on the order of from about 10⁵ to 10¹⁶ of the AAV virions, such as 10⁸ to 10¹⁴ AAV virions, such as AAV6 virions. The dose, of course, depends on the efficiency of transduction, the disclosed promoter that is selected, the stability of the message and the protein encoded thereby, and clinical factors. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ AAV virions in a single dose, or two, four, five, six or more doses that collectively result in delivery of, e.g., 10⁵ to 10¹⁶ AAV virions. One of skill in the art can readily determine an appropriate number of doses to administer.

In some embodiments, the AAV is administered at a dose of about 1×10¹¹ to about 1×10¹⁴ viral particles (vp)/kg. In some examples, the AAV is administered at a dose of about 1×10¹² to about 8×10¹³ vp/kg. In other examples, the AAV is administered at a dose of about 1×10¹³ to about 6×10¹³ vp/kg. In specific non-limiting examples, the AAV is administered at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the AAV is administered at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the AAV is administered at a dose of about 1×1012 vp/kg. The AAV can be administered in a single dose, or in multiple doses (such as 2, 3, 4, 5, 6, 7, 8, 9 or 10 doses) as needed for the desired therapeutic results, such as the production of β cells and/or treatment of type 1 diabetes. The AAV can be an AAV6.

Pharmaceutical compositions include sufficient genetic material to produce a therapeutically effective amount of MAFA and PDX1. In some embodiments, AAV virions will be present in the subject compositions in an amount sufficient to provide a therapeutic effect, such as the production of pancreatic beta cells and/or the treatment of diabetes when given in one or more doses.

AAV virions can be provided as lyophilized preparations and diluted in a stabilizing compositions for immediate or future use. Alternatively, the AAV virions can be provided immediately after production and stored for future use.

The pharmaceutical compositions can contain the vector, such as the AAV vector, and/or virions, and a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol.

Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

In some embodiments, the excipients confer a protective effect on the AAV virion such that loss of AAV virions, as well as transduceability resulting from formulation procedures, packaging, storage, transport, and the like, is minimized. These excipient compositions are therefore considered “virion-stabilizing” in the sense that they provide higher AAV virion titers and higher transduceability levels than their non-protected counterparts, as measured using standard assays, see, for example, Published U.S. Application No. 2012/0219528, incorporated herein by reference. These Compositions therefore demonstrate “enhanced transduceability levels” as compared to compositions lacking the particular excipients described herein, and are therefore more stable than their non-protected counterparts.

Exemplary excipients that can used to protect the AAV virion from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, preferably, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients present in any of the disclosed compositions varies and is readily determined by one of skill in the art. For example, a protein excipient, such as BSA, if present, will can be present at a concentration of between 1.0 weight (wt.) % to about 20 wt. %, preferably 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. If a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous virion-stabilizing formulation comprises a carbohydrate, such as sorbitol, at a concentration of between 0.1 wt. % to about 10 wt. %, such as between about 1 wt. % to about 5 wt. %, and a detergent, such as a sorbitan ester (TWEEN®) at a concentration of between about 0.05 wt. % and about 5 wt. %, such as between about 0.1 wt. % and about 1 wt. %. Virions are generally present in the composition in an amount sufficient to provide a therapeutic effect when given in one or more doses, as defined above.

The pharmaceutical compositions can include a contrast dye is administered in addition to the viral vector, such an adenoviral vector, including a disclosed glucagon promoter operably linked to a heterologous nucleic acid molecule encoding PDX1 and MAFA. The contrast dye can be a low-osmolar low-viscosity non-ionic dye, a low-viscosity high-osmolar dye, or a dissociable high-viscosity dye. In specific non-limiting examples the dye is Iopromid, Ioglicinate, or Ioxaglinate. Thus, provided herein is a pharmaceutical composition including a) an adeno-associated virus vector, such as AAV6, comprising a disclosed promoter operably linked to a nucleic acid molecule encoding PDX1 and a nucleic acid encoding MAFA, optionally wherein the vector does not encode Ngn3; b) a buffer; and c) a contrast dye for endoscopic retrograde cholangiopancreatography. In some embodiments, the pharmaceutical composition does not include a nucleic acid encoding Ngn3. Any of the AAV vectors disclosed herein, including any of the disclosed promoters, can be included in this composition. The AAV vector can be encapsulated in a virion. The AAV vector can be an AAV6 vector. The composition can be formulated for administration to the pancreatic duct.

The disclosed pharmaceutical compositions including a viral vector, such an adenoviral vector or AAV vector, can be delivered to humans or other animals by any means, including orally, intravenously, intramuscularly, intraperitoneally, intranasally, intradermally, intrathecally, subcutaneously, via inhalation or via suppository. In one non-limiting example, the composition is administered into the pancreatic duct of a subject in vivo.

One exemplary method for intraductal administration is Endoscopic Retrograde Cholangiopancreatography (ERCP). ERCP is an endoscopic technique that involves the placement of a side-viewing instrument (generally either an endoscope or duodenoscope) within the descending duodenum. The procedure eliminates the need for invasive surgical procedures for administration to the pancreatic duct.

In an ERCP procedure, the patient will generally lie on their side on an examining table. The patient will then be given medication to help numb the back of the patient's throat, and a sedative to help the patient relax during the examination. The patient then swallows the endoscope. The thin, flexible endoscope is passed carefully through the alimentary canal of the patient. The physician guides the endoscope through the patient's esophagus, stomach, and the first part of the small intestine known as the duodenum. Because of the endoscope's relatively small diameter, most patients can tolerate the unusualness of having the endoscope advanced through the opening of their mouth.

The physician stops the advancement of the endoscope when the endoscope reaches the junction where the ducts of the biliary tree and pancreas open into the duodenum. This location is called the papilla of Vater, or also commonly referred to as the ampulla of Vater. The papilla of Vater is a small mound of tissue looking and acting similarly to a nipple. The papilla of Vater emits a substance known as bile into the small intestine, as well as pancreatic secretions that contain digestive enzymes. Bile is a combination of chemicals made in the liver and is necessary in the act of digestion. Bile is stored and concentrated in the gallbladder between meals. When digestive indicators stimulate the gallbladder, however, the gallbladder squeezes the bile through the common bile duct and subsequently through the papilla of Vater. The pancreas secretes enzymes in response to a meal, and the enzymes help digest carbohydrates, fats, and proteins.

The patient will be instructed (or manually maneuvered) to lie flat on their stomach once the endoscope reaches the papilla of Vater. For visualization or treatment within the biliary tree, the distal end of the endoscope is positioned proximate the papilla of Vater. A catheter is then advanced through the endoscope until the distal tip of the catheter emerges from the opening at the endoscope's distal end. The distal end of the catheter is guided through the endoscope's orifice to the papilla of Vater (located between the sphincter of Oddi) leading to the common bile duct and the pancreatic duct. In the case of pancreas-specific delivery of reagents, the pancreatic duct proper can be entered.

ERCP catheters can be constructed from Teflon, polyurethane and polyaminde. ERCP catheters also can also be constructed from resin comprised of nylon and PEBA (see U.S. Pat. No. 5,843,028), and can be construed for use by a single operator (see U.S. Pat. No. 7,179,252). At times, a spring wire guide may be placed in the lumen of the catheter to assist in cannulation of the ducts. A stylet, used to stiffen the catheter, must first be removed prior to spring wire guide insertion.

A dual or multi-lumen ERCP catheter in which one lumen could be utilized to accommodate the spring wire guide or diagnostic or therapeutic device, and in which a second lumen could be utilized for contrast media and/or dye infusion and or for administration of a pharmaceutical composition including a viral vector, such an adenoviral vector, that includes a disclosed promoter, such as operably linked to a heterologous nucleic acid molecule encoding PDX1 and MAFA. In some embodiments, a contrast dye is administered to the subject.

A contrast dye can be included in a pharmaceutical composition. The contrast dye can be a low-osmolar low-viscosity non-ionic dye, a low-viscosity high-osmolar dye, or a dissociable high-viscosity dye. In specific non-limiting examples the dye is Topromid, Toglicinate, or Toxaglinate. Endoscopes have been designed for the delivery of more than one liquid solution, such as a first liquid composition including a viral vector, such an adenoviral vector, and a second liquid composition including dye, see U.S. Pat. No. 7,597,662, which is incorporated herein by reference. Thus, the pharmaceutical composition including a viral vector, such an adenoviral vector, and the dye can be delivered in the same or separate liquid compositions. Methods and devices for using biliary catheters for accessing the biliary tree for ERCP procedures are disclosed in U.S. Pat. Nos. 5,843,028, 5,397,302, 5,320,602, which are incorporated by reference herein.

In additional examples, the vector is administered using a viral infusion technique into a pancreatic duct. Suitable methods are disclosed, for example, in Guo et al. Laboratory Invest. 93: 1241-1253, 2013, incorporated by reference herein.

### EXAMPLES

Recombinant nucleic acid molecules were produced wherein the alpha cell glucagon promotor was used to drive transcription factors PDX1 and MAFA via AAV serotype 6 vectors to reprogram alpha cells into insulin producing cells and reverse diabetes. This gene deliver approach was used to investigate specific pancreatic alpha cell expression of PDX1 and MAFA genes by AAV6 mediation to determine whether gene transduction can normalize blood glucose levels and whether reprogrammed insulin producing alpha cells can reverse diabetes. Both human and mouse glucagon promoters to drive PDX1 and MAFA genes were evaluated in this study.

### Example 1

### Human Promoters

The human glucagon gene is over 85% percent identical to the mouse, rat, bovine and hamster genes. In fact, the TATA-box and CAAT-box are located at −20 to −26 and −62 to −65 promoter regions, respectively, and has the ability to drive gene expression. Length variations of human glucagon promoter were screened and the 592 bp and 660 bp human glucagon promoters were selected to drive gene expression for the in vitro and in vivo experiments. (FIG. 1).

Human glucagon promoter 592bp
SEQ ID NO: 1
GGGGTTTAATCAATATCCTTAAATTTTCCACAAACATAACATAAATAAAC
TCCACGTTGTGAGGAAGAGAGGATTTTTAATACATATGTGTTGAATGAAT
GATCATTATTTAGATAAATGAATGACTGAAGTGATTGTTATATTCAGGTA
AATTCATCATGGCTAGGTAGCAAACCAAAGACTTGTAAGAACCTCAAATG
AGGACATGCACAAAACAGGGATGGCCATGGGCTACGTAATTTCAAGGTCT
TTTGTCTTCAACGTCAAAATTCACTTTAGAGAACTTAAGTGATTTTCATG
CGTGATTGAAAGTAGAAGGTGGATTTCCAAGCTGCTCTCTCCATTCCCAA
CCAAAAAAAAAAAAAAAAGATACAAGAGTGCATAAAAAGTTTCCAGGTCT
CTAAGGTCTCTCACCCAATATAAGCATAGAATGCAGATGAGCAAAGTGAG
TGGGAGAGGGAAGTCATTTGTAACAAAAACTCATTATTTACAGATGAGAA
ATTTATATTGTCAGCGTAATATCTGTGAGGCTAAACAGAGCTGGAGAGTA
TATAAAAGCAGTGCGCCTTGGTGCAGAAGTACAGAGCTTAGG
Human glucagon promoter 660bp
SEQ ID NO: 2
TGGATGTTTAATCTTCATTTTGCTCCATCCTTTCTGCCTGAATTCCATTT
ATTAAAACAGAACACATAGGGGTTTAATCAATATCCTTAAATTTTCCACA
AACATAACATAAATAAACTCCACGTTGTGAGGAAGAGAGGATTTTTAATA
CATATGTGTTGAATGAATGATCATTATTTAGATAAATGAATGACTGAAGT
GATTGTTATATTCAGGTAAATTCATCATGGCTAGGTAGCAAACCAAAGAC
TTGTAAGAACCTCAAATGAGGACATGCACAAAACAGGGATGGCCATGGGC
TACGTAATTTCAAGGTCTTTTGTCTTCAACGTCAAAATTCACTTTAGAGA
ACTTAAGTGATTTTCATGCGTGATTGAAAGTAGAAGGTGGATTTCCAAGC
TGCTCTCTCCATTCCCAACCAAAAAAAAAAAAAAAAGATACAAGAGTGCA
TAAAAAGTTTCCAGGTCTCTAAGGTCTCTCACCCAATATAAGCATAGAAT
GCAGATGAGCAAAGTGAGTGGGAGAGGGAAGTCATTTGTAACAAAAACTC
ATTATTTACAGATGAGAAATTTATATTGTCAGCGTAATATCTGTGAGGCT
AAACAGAGCTGGAGAGTATATAAAAGCAGTGCGCCTTGGTGCAGAAGTAC
AGAGCTTAGG

### Example 2

### Mouse Promoters

In the mouse glucagon gene promoter region, the TATA-box and CAAT-box are located at −20 to −24 and −64 to −67 promoter regions, respectively, and has the ability to drive gene expression. The 515 bp mouse glucagon promoter was selected to drive gene expression for the in vitro and in vivo experiments. (FIG. 2).

Mouse glucagon promoter 515bp
SEQ ID NO: 3
TTGACATTTAAAATCTCCCCACAAAGAGAATACAAATTTATTCCATACAA
TGTGGATGAGTGGGTTATTGATATGTGTGTACTGGGTGAGTGATTTGTTT
GCCTAGATGAATGATCTATCACTTATTCAGGAACTCAGGATATAGCCAAA
TACCAAATCAAGGGATAAGACCCTCAAATGAGACTAGGCTCATTTGACGT
CAAAATTCACTTGAGAGAACTTTAGCAGTTTTTCGTGCCTGACTGAGACC
GAAGGGTGGATCTCCAAACTGCCCTTTCCATTCCCAAACAGAAAGGCACA
AGAGTAAATAAAATGTTTCCGGGCCTCTGCGGTCTCAACCCGGTATCAGC
GTAAAAAGCAGATGAGCAAAGTGAGTGGGCGAGTGAAATCATTTGAACAA
AACCCCATTATTTACAGATGAGAAATTTATATTGTCAGCGTAATATCTGC
AAGGCTAAACAGCCTGGAGAGCATATAAAAGCACAGCACCCTGGTGCAGA
AGGGCAGAGCTTGGG

### Example 3

### Validation of Glucagon Promoters by Transfection in Human Alpha Cell Line and Mouse Alpha-TC Cell Line

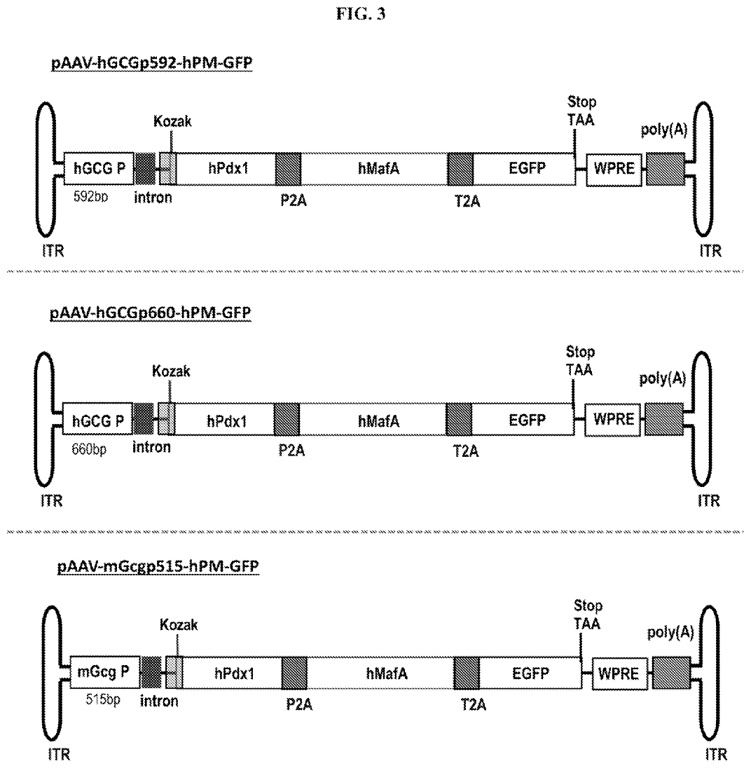

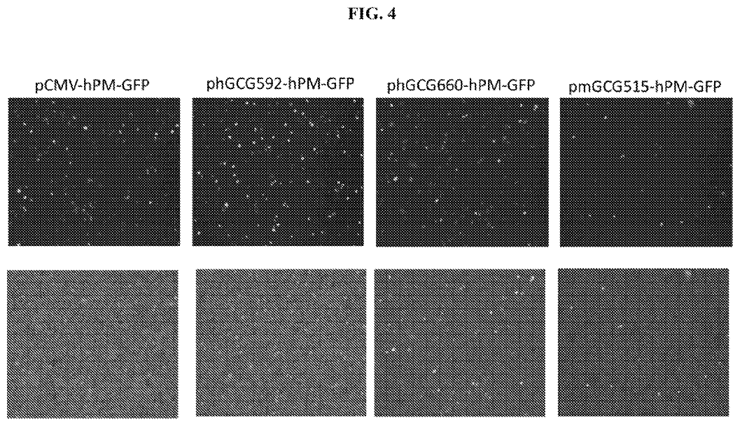

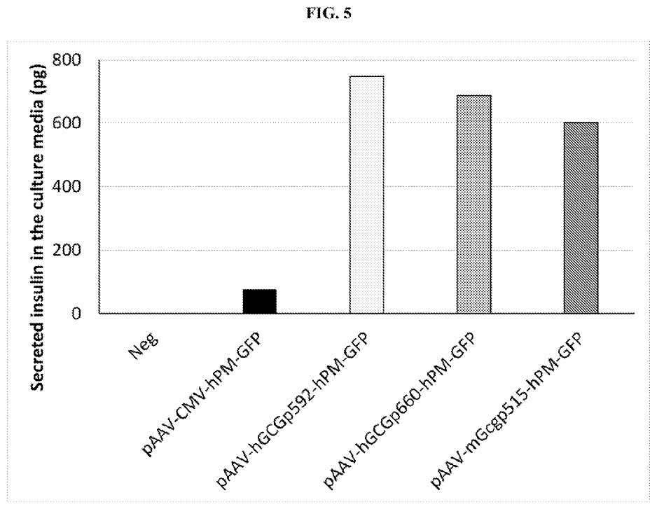

The pAAV-CMV-hPDX1-hMAFA-GFP plasmid was modified to include the human glucagon promoter 592 bp or 660 bp, or mouse glucagon promoter 515 bp to replace the CMV promoter (FIG. 3). The glucagon promoter drives PDX1 and MAFA open reading frames (ORF) and GFP tag was connected with 2A peptide sequences. The transduction efficiency in human alpha cells was determined by GFP (FIG. 4). The alpha to beta conversion was evaluated by insulin ELISA with cultured alpha cells media after transfection. (FIG. 5).

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A recombinant nucleic acid molecule comprising a glucagon promoter operably linked to a nucleic acid molecule encoding a heterologous protein, wherein the glucagon promoter consists essentially of:
a) a nucleotide sequence at least 95% identical to SEQ ID NO: 1, that functions as a promoter;
b) the nucleotide sequence of SEQ ID NO: 1;
c) a nucleotide sequence at least 95% identical to SEQ ID NO: 2, that functions as a promoter;
d) the nucleotide sequence of SEQ ID NO: 2;
e) a nucleotide sequence at least 95% identical to SEQ ID NO: 3, that functions as a promoter; or
f) the nucleotide sequence of SEQ ID NO: 3.

2. The recombinant nucleic acid molecule of claim 1, wherein the glucagon promoter consists of:
a) the nucleotide sequence at least 95% identical to SEQ ID NO: 1, that functions as a promoter;
b) the nucleotide sequence of SEQ ID NO: 1;
c) the nucleotide sequence at least 95% identical to SEQ ID NO: 2, that functions as a promoter;
d) the nucleotide sequence of SEQ ID NO: 2;
e) the nucleotide sequence at least 95% identical to SEQ ID NO: 3, that functions as a promoter; or
f) the nucleotide sequence of SEQ ID NO: 3.

3. The recombinant nucleic acid molecule of claim 1, wherein the glucagon promoter consists of:
a) the nucleotide sequence of SEQ ID NO: 1;
b) the nucleotide sequence of SEQ ID NO: 2; or
c) the nucleotide sequence of SEQ ID NO: 3.

4. A vector comprising the nucleic acid molecule of claim 1.

5. The vector of claim 4, wherein the vector is a viral vector.

6. The vector of claim 5, wherein the viral vector is a lentivirus vector, an adenovirus vector or an adeno-associated virus (AAV) vector.

7. The vector of claim 6, wherein the vector is an AAV6 vector.

8. A host cell transformed with the recombinant nucleic acid molecule of claim 1, comprising the recombinant nucleic acid molecule.

9. The host cell of claim 8, wherein the host cell is a mammalian host cell.

10. The host cell of claim 9, wherein the mammalian host cell is a human host cell or a murine host cell.

11. (canceled)

12. The host cell of claim 8, wherein the host cell is an alpha cell.

13. A method of producing a protein in a host cell, comprising:
transforming the host cell with an effective amount of the vector of claim 4, thereby producing the protein in the host cell.

14. The method of claim 13, wherein the host cell is a mammalian host cell.

15. The method of claim 14, wherein the host cell is a human host cell or a murine host cell.

16. (canceled)

17. The recombinant nucleic acid molecule of claim 1, a vector comprising the nucleic acid molecule, a host cell transformed with the recombinant nucleic acid molecule, wherein the heterologous protein is Pancreas duodenal homeobox protein (PDX1) and/or Musculoaponeurotic fibrosarcoma oncogene homolog A (MAFA).

18. A composition comprising an effective amount of recombinant nucleic acid molecule of claim 1, or a vector comprising the nucleic acid molecule; and a pharmaceutically acceptable carrier.

19. The composition of claim 18, wherein the heterologous protein is PDX1 or MAFA.

20. The composition of claim 18, further comprising a contrast dye for endoscopic retrograde cholangiopancreatography.

21. The composition of claim 20, wherein the contrast dye is wherein the contrast dye is a low-osmolar low-viscosity non-ionic dye, a low-viscosity high-osmolar dye, or a dissociable high-viscosity dye.

22. The composition of claim 20, wherein the contrast dye is Iopromid, Ioglicinate, or Ioxaglinate.

23. A method of producing pancreatic beta cells in a subject, comprising
administering to the subject the vector of claim 4, wherein the glucagon promoter is operably linked to a nucleic acid molecule encoding Pancreas duodenal homeobox protein (PDX)1 and a nucleic acid molecule encoding Musculoaponeurotic fibrosarcoma oncogene homolog A (MAFA), wherein the vector does not encode Neurogenin 3 (Ngn3), wherein the subject is not administered any other nucleic acid encoding Ngn3, wherein the vector is administered intraductally into a pancreatic duct of the subject,
thereby inducing alpha cells to transdifferentiate into pancreatic beta cells in the subject.

24. The method of claim 23, wherein the nucleic acid sequence encoding PDX1 and the nucleic acid sequence encoding MAFA are linked using a connector.

25. The method of claim 24, wherein the connector is a 2A connector.

26. The method of claim 23, wherein intraductally administering comprises the use of endoscopic retrograde cholangiopancreatography (ERCP).

27. The method of claim 23, wherein the subject is not administered an immunosuppressive agent.

28. The method of any claim 23, wherein the subject is a human.

29. The method of claim 28, wherein the subject has type I diabetes.


SEQUENCE LISTING (18 sequences)

SEQ ID NO: 1 - DNA, 592 bp, Homo sapiens
  source 1..592: mol_type = genomic DNA
ggggtttaatcaatatccttaaattttccacaaacataacataaataaactccacgttgtgaggaagagaggatttttaatacatatgtgttgaatgaatgatcattatttagataaatgaatgactgaagtgattgttatattcaggtaaattcatcatggctaggtagcaaaccaaagacttgtaagaacctcaaatgaggacatgcacaaaacagggatggccatgggctacgtaatttcaaggtcttttgtcttcaacgtcaaaattcactttagagaacttaagtgattttcatgcgtgattgaaagtagaaggtggatttccaagctgctctctccattcccaaccaaaaaaaaaaaaaaaagatacaagagtgcataaaaagtttccaggtctctaaggtctctcacccaatataagcatagaatgcagatgagcaaagtgagtgggagagggaagtcatttgtaacaaaaactcattatttacagatgagaaatttatattgtcagcgtaatatctgtgaggctaaacagagctggagagtatataaaagcagtgcgccttggtgcagaagtacagagcttagg

SEQ ID NO: 2 - DNA, 660 bp, Homo sapiens
  source 1..660: mol_type = genomic DNA
tggatgtttaatcttcattttgctccatcctttctgcctgaattccatttattaaaacagaacacataggggtttaatcaatatccttaaattttccacaaacataacataaataaactccacgttgtgaggaagagaggatttttaatacatatgtgttgaatgaatgatcattatttagataaatgaatgactgaagtgattgttatattcaggtaaattcatcatggctaggtagcaaaccaaagacttgtaagaacctcaaatgaggacatgcacaaaacagggatggccatgggctacgtaatttcaaggtcttttgtcttcaacgtcaaaattcactttagagaacttaagtgattttcatgcgtgattgaaagtagaaggtggatttccaagctgctctctccattcccaaccaaaaaaaaaaaaaaaagatacaagagtgcataaaaagtttccaggtctctaaggtctctcacccaatataagcatagaatgcagatgagcaaagtgagtgggagagggaagtcatttgtaacaaaaactcattatttacagatgagaaatttatattgtcagcgtaatatctgtgaggctaaacagagctggagagtatataaaagcagtgcgccttggtgcagaagtacagagcttagg

SEQ ID NO: 3 - DNA, 515 bp, Mus musculus
  source 1..515: mol_type = genomic DNA
ttgacatttaaaatctccccacaaagagaatacaaatttattccatacaatgtggatgagtgggttattgatatgtgtgtactgggtgagtgatttgtttgcctagatgaatgatctatcacttattcaggaactcaggatatagccaaataccaaatcaagggataagaccctcaaatgagactaggctcatttgacgtcaaaattcacttgagagaactttagcagtttttcgtgcctgactgagaccgaagggtggatctccaaactgccctttccattcccaaacagaaaggcacaagagtaaataaaatgtttccgggcctctgcggtctcaacccggtatcagcgtaaaaagcagatgagcaaagtgagtgggcgagtgaaatcatttgaacaaaaccccattatttacagatgagaaatttatattgtcagcgtaatatctgcaaggctaaacagcctggagagcatataaaagcacagcaccctggtgcagaagggcagagcttggg

SEQ ID NO: 4 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 5 - protein, 359 aa, Mus musculus
MAAELAMGAELPSSPLAIEYVNDFDLMKFEVKKEPPEAERFCHRLPPGSLSSTPLSTPCSSVPSSPSFCAPSPGTGGGAGGGGSAAQAGGAPGPPSGGPGTVGGASGKAVLEDLYWMSGYQHHLNPEALNLTPEDAVEALIGSGHHGAHHGAHHPAAAAAYEAFRGQSFAGGGGADDMGAGHHHGAHHTAHHHHSAHHHHHHHHHHGGSGHHGGGAGHGGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGGAGGAGFPREPSPAQAGPGAAKGAPDFFL

SEQ ID NO: 6 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 7 - protein, 284 aa, Mus musculus
MNSEEQYYAATQLYKDPCAFQRGPVPEFSANPPACLYMGRQPPPPPPPQFTSSLGSLEQGSPPDISPYEVPPLASDDPAGAHLHHHLPAQLGLAHPPPGPFPNGTEPGGLEEPNRVQLPFPWMKSTKAHAWKGQWAGGAYTAEPEENKRTRTAYTRAQLLELEKEFLFNKYISRPRRVELAVMLNLTERHIKIWFQNRRMKWKKEEDKKRSSGTPSGGGGGEEPEQDCAVTSGEELLAVPPLPPPGGAVPPGVPAAVREGLLPSGLSVSPQPSSIAPLRPQEPR

SEQ ID NO: 8 - DNA, 48 bp, synthetic construct
  source 1..48: mol_type = other DNA
cgcgccaagcgcggctccggcgccaccaacttctccctgctgaagcag

SEQ ID NO: 9 - DNA, 81 bp, synthetic construct
  source 1..81: mol_type = other DNA
cgcgccaagcgcggctccggccagtgcaccaactacgccctgctgaagctggccggcgacgtggagtccaaccccggcccc

SEQ ID NO: 10 - DNA, 75 bp, synthetic construct
  source 1..75: mol_type = other DNA
cgcgccaagcgcggctccggcgagggcagaggaagtcttctaacatgcggtgacgtggaggagaatcccggccct

SEQ ID NO: 11 - protein, 214 aa, Homo sapiens
MAPHPLDALTIQVSPETQQPFPGASDHEVLSSNSTPPSPTLIPRDCSEAEVGDCRGTSRKLRARRGGRNRPKSELALSKQRRSRRKKANDRERNRMHNLNSALDALRGVLPTFPDDAKLTKIETLRFAHNYIWALTQTLRIADHSFYGPEPPVPCGELGSPGGGSNGDWGSIYSPVSQAGNLSPTASLEEFPGLQVPSSPSYLLPGALVFSDFL

SEQ ID NO: 12 - DNA, 38 bp, Homo sapiens
  source 1..38: mol_type = genomic DNA
acaaaaactcattatttacagatgagaaatttatattg

SEQ ID NO: 13 - DNA, 23 bp, Homo sapiens
  source 1..23: mol_type = genomic DNA
caagagtgcataaaaagtttcca

SEQ ID NO: 14 - DNA, 33 bp, Homo sapiens
  source 1..33: mol_type = genomic DNA
agtgattttcatgcgtgattgaaagtagaaggt

SEQ ID NO: 15 - DNA, 27 bp, Homo sapiens
  source 1..27: mol_type = genomic DNA
tttgtcttcaacgtcaaaattcacttt

SEQ ID NO: 16 - DNA, 46 bp, Mus musculus
  source 1..46: mol_type = genomic DNA
gaacaaaaccccattatttacagatgagaaatttatattgtcagcg

SEQ ID NO: 17 - DNA, 32 bp, Mus musculus
  source 1..32: mol_type = genomic DNA
agtttttcgtgcctgactgagaccgaagggtg

SEQ ID NO: 18 - DNA, 25 bp, Mus musculus
  source 1..25: mol_type = genomic DNA
aggctcatttgacgtcaaaattcac